(19) United States
(12) Patent Application Publication
(10) Pub. No.: US 2021/0401701 A1
(43) Pub. Date: Dec. 30, 2021

(54) ANTIDANDRUFF COMPOSITION

(71) Applicant: Conopco, Inc., d/b/a UNILEVER, Trumbull, CT (US)

(72) Inventors: Joshua Ryan Carew, Chester (GB); Adam James Simkins, Dobcross (GB); Amy Elizabeth Vanvelsor, Seymour, CT (US)

(73) Assignee: Conopco, Inc., d/b/a UNILEVER, Trumbull, CT (US)

(21) Appl. No.: 16/910,517

(22) Filed: Jun. 24, 2020

(51) Int. Cl. A61K 8/368; A61Q 5/00; A61K 8/92; A61K 8/96; A61K 8/27; A61K 8/23; A61K 8/44; A61K 8/55; A61K 8/06

(52) U.S. Cl. CPC A61K 8/368; A61Q 5/006; A61K 8/922; A61K 8/965; A61K 8/06; A61K 8/23; A61K 8/44; A61K 8/55; A61K 8/27

(57) ABSTRACT

An antidandruff composition includes about 0.1 to about 1% by weight of a natural oil containing a mixture of C12 and C14 alkyl groups derived from coconut oil; about 6% by weight of a non-ionic surfactant having at least 16 carbon atoms; about 2% by weight of an antidandruff agent; about 1.5% by weight of a cationic surfactant; about 1.5% by weight of a quaternary ammonium salt; and at least 75% by weight of water. A method of making an antidandruff composition includes combining a natural oil containing a mixture of C12 and C14 alkyl groups, a non-ionic surfactant having at least 16 carbon atoms, an antidandruff agent, a cationic surfactant, a quaternary ammonium salt, and water, forming the antidandruff composition.

### FIELD OF THE INVENTION

Disclosed herein is an antidandruff composition. The antidandruff composition can include a natural oil containing a mixture of C12 and C14 alkyl groups, a non-ionic surfactant having at least 16 carbon atoms, an antidandruff agent, a cationic surfactant, a quaternary ammonium salt, and water.

### BACKGROUND OF THE INVENTION

Hair care compositions generally provide cleansing or conditioning benefits or a combination thereof. Such compositions typically comprise one or more cleansing surfactants which generally aid in cleaning the hair and the scalp from undesirable soil, particles and fatty matter. A conditioning benefit is achieved by including one or more conditioning agents in the hair care composition. A conditioning benefit is delivered with an oily material being deposited onto the hair resulting in the formation of a film, which makes the hair easier to comb when wet and more manageable when dry.

Additionally, antidandruff benefit has been provided through hair care compositions. Dandruff is an issue that affects many people globally. The condition is manifested by the shedding of clumps of dead skin cells from the scalp. These are white in color and provide an aesthetically displeasing appearance. A factor that contributes to dandruff is certain members of the Malassezia yeasts. To combat these, antidandruff products have included certain antidandruff agents which have anti-fungal activity.

Depending on the active ingredient used in the compositions, discoloration can occur, e.g., turning of the composition from white to a different color. For example, the use of salicylic acid in some compositions, can affect the color of the composition and turn an otherwise white composition to pink.

It is increasingly desired to provide an antidandruff composition containing fewer overall ingredients, without the inclusion of silicone, that still provides the desired antidandruff and conditioning benefits also without suffering from discoloration.

### SUMMARY OF THE INVENTION

An antidandruff composition, comprises: about 0.1 to about 1% by weight of a natural oil containing a mixture of C12 and C14 alkyl groups; about 6% by weight of a non-ionic surfactant having at least 16 carbon atoms; about 2% by weight of an antidandruff agent; about 1.5% by weight of a cationic surfactant; about 1.5% by weight of a quaternary ammonium salt; and at least 75% by weight of water.

A method of making an antidandruff composition, comprises: combining about 0.1 to about 1% by weight of a natural oil containing a mixture of C12 and C14 alkyl groups, about 6% by weight of a non-ionic surfactant having at least 16 carbon atoms, about 2% by weight of an antidandruff agent, about 1.5% by weight of a cationic surfactant, about 1.5% by weight of a quaternary ammonium salt, and at least 75% by weight of water, forming the antidandruff composition.

### DETAILED DESCRIPTION OF THE INVENTION

Disclosed herein is an antidandruff composition. The antidandruff composition contains fewer ingredients, specifically, contains no silicone, as compared to typical antidandruff compositions. The presently disclosed antidandruff composition contains fewer ingredients without suffering from any discoloration. The antidandruff composition can include about 0.1 to about 1% by weight of a natural oil containing a mixture of C12 and C14 alkyl groups, about 6% by weight of a nonionic surfactant having at least 16 carbon atoms, about 1.5% by weight of a cationic surfactant, about 1.5% by weight of a quaternary ammonium salt, about 2% by weight of an antidandruff agent, and at least 75% by weight of water. It was unexpectedly discovered that the antidandruff compositions disclosed herein do not suffer from discoloration. Without wishing to be bound by theory, it is believed that changing the processing temperature during the emulsion process of making the conditioner stopped the formation of colored bodies within the conditioner emulsion.

The natural oil can be derived from any natural oil, including but not limited to, coconut oil (containing lauric acid), olive oil (containing a mixture of oleic, stearic, linoleic, palmitic, and arachidic acid), jojoba oil, castor oil (containing rinoleic acid), cocoa butter, shea butter (containing a mixture of oleic, stearic, linoleic, palmitic, and arachidic acid), argan oil (containing a mixture of linoleic acid, vitamin E, and oleic acid), avocado oil, tea tree oil, or a combination thereof. The natural oil can be coconut oil. The natural oil can be present in an amount of about 0.01 to about 5% by weight, for example, 0.05 to about 2.5% by weight, for example, about 0.07 to about 2.% by weight, for example, 0.09 to about 1.5% by weight, for example, about 0.1 to about 1% by weight, for example, about 0.2 to about 0.9% by weight.

Nonionic surfactants can be used in the antidandruff composition. When used, nonionic surfactants can be used at levels as low as 0.5, 1, 1.5, or 2% by weight and at levels as high as 6, 8, 10, or 12% by weight. In the antidandruff composition disclosed herein, the nonionic surfactant can be present in an amount of about 3% by weight, for example, about 5% by weight, for example, about 6% by weight, for example, about 7% by weight, for example, about 8% by weight.

The nonionic surfactants which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic surfactant compounds are alkyl (C₆-C₂₂) phenols, ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other nonionic surfactants include long chain tertiary amine oxides, long chain tertiary phosphine oxides, dialkyl sulphoxides, and the like. Preferred nonionic surfactants have at least 16 carbon atoms. For example, nonionic surfactants can comprise a combination of C16 and C18, for example, the nonionic surfactants can contain 25% C16 and 75% C18, for example, 30% C16 and 70% C18, for example, 35% C16 and 65% C18. For example, the nonionic surfactant can comprise cetearyl alcohol with the listed carbon atoms.

In an aspect, nonionic surfactants can include fatty acid/alcohol ethoxylates having the following structures a) HOCH₂(CH₂)ₛ(CH₂CH₂O)_{c} H or b) HOOC(CH₂)ᵥ(CH₂CH₂O)_{d} H; where s and v are each independently an integer up to 18; and c and d are each independently an integer from 1 or greater. In an aspect, s and v can be each independently 6 to 18; and c and d can be each independently 1 to 30. Other options for nonionic surfactants include those having the formula HOOC(CH₂)ᵢ—CH═CH—(CH₂)ₖ(CH₂CH₂O)_{z}H, where i and k are each independently 5 to 15; and z is 5 to 50. In another aspect, i and k are each independently 6 to 12; and z is 15 to 35.

The nonionic surfactant may also include a sugar amide, such as a polysaccharide amide. Specifically, the surfactant may be one of the lactobionamides described in U.S. Pat. No. 5,389,279 to Au et al., entitled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Illustrative examples of nonionic surfactants that can optionally be used in the antidandruff compositions disclosed herein include, but are not limited to, polyglycoside, cetyl alcohol, decyl glucoside, lauryl glucoside, octaethylene glycol monododecyl ether, n-octyl beta-d-thioglucopyranoside, octyl glucoside, oleyl alcohol, polysorbate, sorbitan, stearyl alcohol, or a combination thereof.

In an aspect, cationic surfactants may optionally be used in the antidandruff composition of the present application.

One class of cationic surfactants includes heterocyclic ammonium salts such as cetyl or stearyl pyridinium chloride, alkyl amidoethyl pyrrylinodium methyl sulfate, and lapyrium chloride.

Tetra alkyl ammonium salts are another useful class of cationic surfactants for use. Examples include cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride.

Still other types of cationic surfactants that may be used are the various ethoxylated quaternary amines and ester quats. Examples include PEG-5 stearyl ammonium lactate (e.g., Genamin KSL manufactured by Clariant), PEG-2 coco ammonium chloride, PEG-15 hydrogenated tallow ammonium chloride, PEG 15 stearyl ammonium chloride, dipalmitoyl ethyl methyl ammonium chloride, dipalmitoyl hydroxyethyl methyl sulfate, stearamidopropyl dimethylamine, and stearyl amidopropyl dimethylamine lactate.

Still other useful cationic surfactants include quaternized hydrolysates of silk, wheat, and keratin proteins, and it is within the scope of the antidandruff composition to use mixtures of the aforementioned cationic surfactants.

If used, cationic surfactants can be present in an amount of 1.0% to 5% by weight of the antidandruff composition, for example, 1.5% by weight, for example, 1.75% by weight, for example, 2.0% by weight, for example, 2.5% by weight, for example, 3.5% by weight, for example, 1.5% by weight to 2.5% by weight, including all ranges subsumed therein.

The antidandruff composition can further include a quaternary ammonium salt.

Quaternary ammonium salts can be included in the composition to impart a desirably slippery feel to the hair after conditioning. Such quaternary ammonium salts include, but are not limited to benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, behentrimonium chloride, domiphen bromide, or a combination thereof. Quaternary ammonium salts can be present in an amount of about 1.5% by weight, for example, about 0.5 to about 3% by weight, for example, about 1 to about 2% by weight, for example, about 1.5% by weight, for example, about 1.75% by weight, for example, about 2% by weight, and any all subranges subsumed therein.

Antidandruff agents used in the antidandruff composition disclosed herein can include metal pyrithione salts e.g., zinc pyrithione, piroctone olamine (octopirox), azoic based anti-fungal agents (e.g. climbazole, azelaic acid), salicylic acid, selenium sulphide, coal tar, or a combination thereof. Antidandruff agents, such as salicylic acid, can be used in an amount of about 2% by weight, for example, about 0.5% to about 5% by weight, for example, about 1% to about 4.5% by weight, for example, about 1.5% to about 3% by weight, for example, about 2% to about 3% by weight.

Optionally, the antidandruff composition disclosed herein can further include a thickening agent. Particularly preferred thickening agents include an anionic polymer-based thickener, preferably, for example, a polyacrylate based polymer or copolymer, or a combination thereof. Polysaccharides can be used. Examples include fibers, starches, natural/synthetic gums and cellulosics. Representative of the starches are chemically modified starches such as sodium hydroxypropyl starch phosphate and aluminum starch octenylsuccinate. Tapioca starch is often preferred, as is maltodextrin. Suitable gums include xanthan, sclerotium, pectin, karaya, arabic, agar, guar (including Acacia senegal guar), carrageenan, alginate and combinations thereof. Desirable cellulosics include hydroxypropyl cellulose, hydroxypropyl methylcellulose, ethylcellulose, sodium carboxy methylcellulose (cellulose gum/carboxymethyl cellulose) and cellulose (e.g. cellulose microfibrils, cellulose nanocrystals or microcrystalline cellulose).

Sources of cellulose microfibrils include secondary cell wall materials (e.g. wood pulp, cotton), bacterial cellulose, and primary cell wall materials. Preferably the source of primary cell wall material is selected from parenchymal tissue from fruits, roots, bulbs, tubers, seeds, leaves and combination thereof; more preferably is selected from citrus fruit, tomato fruit, peach fruit, pumpkin fruit, kiwi fruit, apple fruit, mango fruit, sugar beet, beet root, turnip, parsnip, maize, oat, wheat, peas and combinations thereof; and even more preferably is selected from citrus fruit, tomato fruit and combinations thereof. A most preferred source of primary cell wall material is parenchymal tissue from citrus fruit. Citrus fibers, such as those made available by Herbacel® as AQ Plus can also be used as source for cellulose microfibrils. The cellulose sources can be surface modified by any of the known methods including those described in Colloidal Polymer Science, Kalia et al., “Nanofibrillated cellulose: surface modification and potential applications” (2014), Vol 292, Pages 5-31.

As mentioned, synthetic polymers are effective thickening agents. This category includes crosslinked polyacrylates such as the Carbomers, polyacrylamides such as Sepigel® 305 and taurate copolymers such as Simulgel® EG and Aristoflex® AVC, the copolymers being identified by respective INCI nomenclature as sodium acrylate/sodium acryloyldimethyl taurate and acryloyl dimethyltaurate/vinyl pyrrolidone copolymer. Another preferred synthetic polymer suitable for thickening is an acrylate-based polymer made commercially available by Seppic and sold under the name Simulgel INS100. Calcium carbonate, fumed silica, and magnesium-aluminum-silicate may also be used.

Sodium hydroxypropyl starch phosphate, aluminum starch octenylsuccinate, tapioca starch, maltodextrin, xanthan gum, agar gum, guar gum, carrageenan gum, alginate gum, hydroxypropyl cellulose, hydroxypropyl methylcellulose, ethylcellulose, sodium carboxy methylcellulose, cellulose, polyethylene glycol (e.g., polyethylene glycol diester stearic acid), or a combination thereof are further examples of thickening agents for use in the present antidandruff compositions. Such thickening agents are commercially available from the Dow Chemical Company or the Hallstar Company.

A conditioning agent can be included in the antidandruff composition. Conditioning agents can include occlusives, e.g., petrolatum, dimethicone, and the like; humectants, e.g., glycerin, propylene glycol, sorbitol, and the like; emollients and oils, e.g., trigylcerides, natural oils, lanolin, synthetic esters, and the like; proteins; silicones, e.g., dimethicone, cyclomethicone, amodimethicone, and the like; cationic surfactants, e.g., cetrimonium chloride, stearalkonium chloride, and the like; and polymers, e.g., cationic polymers such as polyquarterniums. When present, the conditioning agent can be present in an amount of 2% by weight to 7% by weight, preferably, 3% by weight to 6% by weight, more preferably, 3.5% by weight to 5% by weight.

Preservatives can desirably be incorporated into antidandruff composition to protect against the growth of potentially harmful microorganisms. Cosmetic chemists are familiar with appropriate preservatives and routinely choose them to satisfy the preservative challenge test and to provide product stability. Traditional preservatives for use include hydantoin derivatives and propionate salts. Preservatives for use are iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin and benzyl alcohol and mixtures thereof. Other preservatives include sodium benzoate, sodium dehydroacetate, chlorophenesin and decylene glycol. Other preservatives include trisodium ethylenediamine disccinate (EDDS), tetra sodium iminodicusscinate (IDS), sodium gluconate, phytic acid, sodium phytate, tricalcium citrate, trisodium dicarboxymethyl alaninate, caproyl/capryloyl anhydro methyl glucamide and water, caprylyl glycol, ethyl lauroyl arginate hydrochloric acid and glycerin, ethyl lauroyl arginate hydrochloric acid and capryl glycol and glycerin, gluconolactone, glyceryl caprylate, lactic acid, p-anisic acid, pentylene glycol, sodium citrate, sorbitan caprylate, butylated hydroxytoluene (BHT), dilauryl thiodipropionate, octadecyl di-t-butyl-4-hydroxyhydrocinnamate, pentaerythrityl tetra-di-t-butyll hydrohydrocinnamate, vitamin E, or a combination thereof. The preservatives should be selected having regard for the use of the composition and possible incompatibilities between the preservatives and other ingredients in the emulsion.

Preservatives are preferably employed in amounts ranging from 0.01% to 2% by weight of the total weight of the composition, including all ranges subsumed therein. Preferred preservatives included, but are not limited to, benzoic acid, levulinic acid, ansic acid, iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin and benzyl alcohol, sodium benzoate, sodium dehydroacetate, chlorophenesin, decylene glycol, or a combination thereof. Also preferred is a preservative system with hydroxyacetophenone alone or in a mixture with other preservatives. Particularly preferred is sodium benzoate. Other preservatives that can be used include benzoin gum, Sapindus mukorossi (soap nut) fruit extract, or a combination thereon.

Additional optional ingredients which may be present in the subject personal antidandruff compositions are, for example: fragrances; sequestering and chelating agents such as tetrasodium ethylenediaminetetraacetate (EDTA), ethane hydroxyl diphosphonate (EHDP), and etidronic acid, aka 1-hydroxyethylidene diphosphonic acid (HEDP), or a combination thereof; coloring agents; opacifiers and pearlizers such as zinc stearate, magnesium stearate, TiO₂, ethylene glycol monostearate (EGMS), ethylene glycol distearate (EGDS) or Lytron 621 (Styrene/Acrylate copolymer) and the like; pH adjusters; antioxidants, for example, butylated hydroxytoluene (BHT) and the like; stabilizers; suds boosters, such as for example, coconut acyl mono- or diethanol amides; ionizing salts, such as, for example, sodium chloride and sodium sulfate, and other ingredients such as are conventionally used in such compositions, such as propylene glycol or dipropylene glycol. The total amount of such additional optional ingredients is typically from 0 to 10% by weight, more particularly from 0.1 to 5% by weight, based on the total weight of the personal antidandruff composition.

The antidandruff composition can additionally include various additives including, but not limited to, colorants, emollients, skin feel agents, hair dyes, styling polymer, silicon oil, cationic polymers, or a combination thereof. Each of these substances may range from about 0.03 to about 5%, preferably between 0.1 and 3% by weight of the total weight of the liquid and composition, including all ranges subsumed therein. For example, colorants can be present in an amount of 5 parts per million (ppm) to 15 ppm, for example, about 15 ppm.

Water preferably makes up 10 to 99% by weight of the liquid and composition, preferably 65 to 95% by weight of the liquid and composition, and more preferably, from 70 to 90% by weight water based on total weight of the liquid and composition, or even 75% by weight, including all ranges subsumed therein. In use, the antidandruff compositions are commonly diluted with water.

A method of making the antidandruff composition can include heating the composition comprising combining about 0.1 to about 1% by weight of a surfactant containing a mixture of C12 and C14 alkyl groups derived from coconut oil, about 6% by weight of a non-ionic surfactant having at least 16 carbon atoms, about 2% by weight of an antidandruff agent, about 1.5% by weight of a cationic surfactant, about 1.5% by weight of a quaternary ammonium salt, and at least 75% by weight of water to a temperature of about 85° C. after which the emulsion process begins. The emulsion mixes for at least 30 minutes. The emulsion temperature can be 75 to 100° C., for example, 80 to 95° C., for example, 85 to 90° C.

The composition may be formulated as an emulsion or a gel.

The following examples are merely illustrative of the adhesive compositions disclosed herein and are not intended to limit the scope hereof.

### EXAMPLES

In the following example, tests were conducted to determine from where the discoloration was occurring and how to prevent the discoloration because it was discovered that after 2 to 4 days in a 45° C. oven, the antidandruff compositions changed in color from white to pink.

Except where noted, the compositions of the examples were made from the following materials listed in Table 1.

TABLE 1

| Material Description | % added |
|---|---|
| Water-Local-Demineralised-MicroTreated | 57.53 |
| Cocos Nucifera (Coconut) Oil 100% | 1.00 |
| Cetearyl Alcohol 30-70 C16-C18 | 8.00 |
| Salicylic Acid | 2.00 |
| Stearamidopropyl dimethylamine | 2.00 |
| Behentrimonium Chloride and DPG | 2.00 |
| Water-Local-Demineralised-MicroTreated | 19.10 |
| Water-Local-Demineralised-MicroTreated | 5.00 |
| Disodium EDTA | 0.10 |
| Sodium Chloride | 0.2 |
| Glycerin Pharmacopeia Grade 99.5% | 1.00 |
| FRAG: Honeysuckle & Watermint 9 | 0.55 |
| Lonicera Japonica (Honeysuckle) Flower | 0.0100 |
| Extract + BG(Pres.) | |
| Sodium Benzoate 33% | 1.51 |

### Example 1

First, the amount of chelating agent i.e., ethylenediaminetetraacetate (EDTA) in the composition was increased to determine if this would prevent the color change. For this test there were 10 samples, 5 with 0.10% Disodium EDTA and 5 with 0.20% Disodium EDTA. Each group had 1 un-fragranced sample and then 4 others each containing a different fragrance oil. After 2-4 days, a color change was observed, so it was determined that amount of chelating agent would not prevent the color change.

### Example 2

In the following example, before each set of samples were made the beaker, blades and any other additional spatulas or utensils were cleaned and sprayed down with isopropyl alcohol.

This example was run to determine if there was contamination occurring from the equipment being used. The salicylic acid commercially available from Novacyl that had been used in all the previous examples was used and the level of EDTA was set at 0.10% since in the previous batches raising the level to 0.20% did not prevent the color change. After the samples were made, they were split 3 ways: was an un-fragranced sample, a fragrance only sample, and a sample with fragrance oil and an antioxidant that was butylated hydroxytoluene (BHT). One sample of each went in the oven (45° C. for 4 days) and one sample was left at room temperature (25° C.). After the 4-day period, all the samples were pink.

Thus, carry over from instruments did not cause the pink issue.

### Example 3

The samples in this example were made to determine if the salicylic acid supplier made a difference. Salicylic acid supplied by Novacyl and Brenntag were both used. Again, ensuring all the instruments were clean, the samples were made and split the same way as the previous examples. There was an un-fragranced sample, a fragrance only sample and a sample with fragrance oil and the antioxidant (BHT). One sample of each went in the oven (45° C. for 4 days) and one sample was left at room temperature (25° C.). When checked after the 4-day period all the samples were pink.

It was therefore unlikely that the salicylic acid supplier was the source of the color change.

### Example 4

After that the water source was tested to determine if it was the source of the color change. For this example, the water source went through an extra purification process. The machine doing the purification was a Milli-Q from Millipore Corporation (serial #F1PA4415OC) which produces ultrapure water. The batch was split the same way as the previous batch: an un-fragranced sample, a fragrance only sample, and a sample with fragrance oil and an antioxidant (BHT). One sample of each went in the oven (45° C. for 4 days) and one sample was left at room temperature (25° C.). When checked after the 4-day period all the samples were pink.

It was therefore unlikely that the water was the source of the color change.

### Example 5

In this example, it was decided to vary the processing temperature to determine if a higher processing temperature could prevent discoloration. The tests were run ensuring all instruments were clean, however distilled water was used and the processing temperature was raised from 63° C. to 85° C. The batch was split into: an un-fragranced sample, a fragrance only sample, and a sample with fragrance oil and an antioxidant (BHT). One sample of each went in the oven (45° C. for 4 days) and one sample was left at room temperature (25° C.). When checked after the 4-day period, these samples were the only ones of all the samples tested that remained white.

It was determined that the higher processing temperature seemingly stops the color change from happening.

Except where otherwise explicitly indicated, all numbers in this description indicating amounts of material or conditions of reaction, physical properties of materials and/or use are to be understood as modified by the word “about.” All amounts are by weight of the final composition, unless otherwise specified.

It should be noted that in specifying any range of concentration or amount, any particular upper concentration can be associated with any particular lower concentration or amount as well as any subranges consumed therein. In that regard, it is noted that all ranges disclosed herein are inclusive of the endpoints, and the endpoints are independently combinable with each other (e.g., ranges of “up to 25% by weight, or, more specifically, 5% by weight to 20% by weight, in inclusive of the endpoints and all intermediate values of the ranges of 5% by weight to 25% by weight, etc.). “Combination is inclusive of blends, mixtures, alloys, reaction products, and the like. Furthermore, the terms “first”, “second”, and the like herein do not denote any order, quantity, or importance, but rather are used to distinguish one element from another. The terms “a” and “an” and “the” herein do not denote a limitation of quantity and are to be construed to cover both the singular and the plural, unless otherwise indicated herein or clearly contradicted by context. The suffix “(s)” as used herein is intended to include both the singular and the plural of the term it modifies, thereby including one or more of the term (e.g., the film(s) includes one or more films). Reference throughout the specification to “one embodiment”, “one aspect”, “another embodiment”, “another aspect”, “an embodiment”, “an aspect” and so forth means that a particular element (e.g., feature, structure, and/or characteristic) described in connection with the embodiment or aspect is included in at least one embodiment or aspect described herein and may or may not be present in other embodiments or aspects. In addition, it is to be understood that the described elements may be combined in any suitable manner in the various embodiments or aspects.

All cited patents, patent applications, and other references are incorporated herein by reference in their entirety. However, if a term in the present application contradicts or conflicts with a term in the incorporated reference, the term from the present application takes precedence over the conflicting term from the incorporated reference. While particular aspects have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

For the avoidance of doubt the word “comprising” is intended to mean “including” but not necessarily “consisting of” or “composed of.” In other words, the listed steps, options, or alternatives need not be exhaustive.

The disclosure of the invention as found herein is to be considered to cover all aspects as found in the claims as being multiply dependent upon each other irrespective of the fact that claims may be found without multiple dependency or redundancy. Unless otherwise specified, numerical ranges expressed in the format “from x to y” are understood to include x and y. In specifying any range of values or amounts, any particular upper value or amount can be associated with any particular lower value or amount. All percentages and ratios contained herein are calculated by weight unless otherwise indicated. The various features of the present invention referred to in individual sections above apply, as appropriate, to other sections mutatis mutandis. Consequently, features specified in one section may be combined with features specified in other sections as appropriate. Any section headings are added for convenience only and are not intended to limit the disclosure in any way.

What is claimed is:

1. An antidandruff composition, comprising:
about 0.1 to about 1% by weight of a natural oil containing a mixture of C12 and C14 alkyl groups;
about 6% by weight of a non-ionic surfactant having at least 16 carbon atoms;
about 2% by weight of an antidandruff agent;
about 1.5% by weight of a cationic surfactant;
about 1.5% by weight of a quaternary ammonium salt; and
at least 75% by weight of water.

2. The antidandruff composition of claim 1, wherein the natural oil comprises coconut oil, olive oil cocoa butter, argan oil, avocado oil, tea tree oil, castor oil, shea butter, jojoba oil, or a combination thereof.

3. The antidandruff composition of claim 1, wherein the non-ionic surfactant comprises a mixture of C16 and C18.

4. The antidandruff composition of claim 3, wherein the non-ionic surfactant comprises 25% C16 and 75% C18.

5. The antidandruff composition of claim 4, wherein the non-ionic surfactant comprises 30% C16 and 70% C18.

6. The antidandruff composition of claim 5, wherein the non-ionic surfactant comprises 35% C16 and 65% C18.

7. The antidandruff composition of claim 1, wherein the non-ionic surfactant comprises polyglycoside, cetyl alcohol, decyl glucoside, lauryl glucoside, octaethylene glycol monododecyl ether, n-octyl beta-d-thioglucopyranoside, octyl glucoside, oleyl alcohol, polysorbate, sorbitan, stearyl alcohol, or a combination thereof.

8. The antidandruff composition of claim 1, wherein the antidandruff agent comprises, zinc pyrithione, salicylic acid, selenium sulphide, coal tar, or a combination thereof.

9. The antidandruff composition of claim 8, wherein the antidandruff agent comprises salicylic acid.

10. The antidandruff composition of claim 1, wherein the cationic surfactant comprises cetyl or stearyl trimethyl ammonium chloride or bromide; hydrogenated palm or tallow trimethylammonium halides; behenyl trimethyl ammonium halides or methyl sulfates; decyl isononyl dimethyl ammonium halides; ditallow (or distearyl) dimethyl ammonium halides, and behenyl dimethyl ammonium chloride, stearamidopropyl dimethylamine, or a combination thereof.

11. The antidandruff composition of claim 1, wherein the quaternary ammonium salt comprises benzalkonium chloride, benzethonium chloride, methylbenzethonium chloride, cetalkonium chloride, cetylpyridinium chloride, cetrimonium, cetrimide, dofanium chloride, tetraethylammonium bromide, didecyldimethylammonium chloride, behentrimonium chloride, domiphen bromide, or a combination thereof.

12. The antidandruff composition of claim 1, further comprising a chelating agent.

13. The antidandruff composition of claim 12, wherein the chelating agent comprises tetrasodium ethylenediaminetetraacetate (EDTA), ethane hydroxyl diphosphonate (EHDP), etidronic acid, aka 1-hydroxyethylidene diphosphonic acid (HEDP), disodium ethylenediaminetetraacetate (EDTA), or a combination thereof.

14. The antidandruff composition of claim 1, further comprising a preservative.

15. The antidandruff composition of claim 14, wherein the preservative comprises benzoic acid, levulinic acid, ansic acid, iodopropynyl butyl carbamate, phenoxyethanol, 1,2-octanediol, hydroxyacetophenone, ethylhexylglycerine, hexylene glycol, methyl paraben, propyl paraben, imidazolidinyl urea, sodium dehydroacetate, dimethyl-dimethyl (DMDM) hydantoin and benzyl alcohol, sodium benzoate, sodium dehydroacetate, chlorophenesin, decylene glycol, or a combination thereof.

16. The antidandruff composition of claim 1, wherein the natural oil is derived from coconut oil.

17. A method of making an antidandruff composition, comprising:
combining about 0.1 to about 1% by weight of a natural oil containing a mixture of C12 and C14 alkyl groups, about 6% by weight of a non-ionic surfactant having at least 16 carbon atoms, about 2% by weight of an antidandruff agent, about 1.5% by weight of a cationic surfactant, about 1.5% by weight of a quaternary ammonium salt, and at least 75% by weight of water, forming the antidandruff composition.

18. The method of claim 17, comprising heating the composition to about 85° C. and forming an emulsion.

19. The method of claim 17, wherein the non-ionic surfactant comprises a mixture of C16 and C18.
